(19) United States
(12) Patent Application Publication
(10) Pub. No.: US 2002/0042589 A1
(43) Pub. Date: Apr. 11, 2002

(54) Medical injection device

(76) Inventor: Thomas Marsoner, Gargnano (IT)

Correspondence Address: WENDEROTH, LIND & PONACK, L.L.P., 2033 K STREET N. W., SUITE 800, WASHINGTON, DC, 20006-1021, US

(21) Appl. No.: 09/988,723

(22) Filed: Nov. 20, 2001

Related U.S. Application Data
(63) Continuation of application No. PCT/AT01/00319, filed on Oct. 5, 2001.

(30) Foreign Application Priority Data
Oct. 5, 2000 (AU) A 1688/2000
May 30, 2001 (AU) A 845/2001

(51) Int. Cl. A61B 17/20; A61M 37/00

(52) U.S. Cl. 604/46; 604/117; 604/265

(57) ABSTRACT

A medical injection device for intracutaneous administration of at least an anaesthetic, comprising a base surface (1), on one side of which a plurality of injectors (2) and in particular injection needles are arranged distributed over the area thereof, wherein the injectors (2) have an injection depth at a maximum as far as the subcutis of the animal or human to be treated, wherein the medical injection device has a disinfecting agent on the side of the base surface (1) on which the injectors (2) are arranged.

The present invention concerns a medical injection device for intracutaneous administration of at least an anaesthetic, comprising a base surface, on one side of which a plurality of injectors and in particular injection needles are arranged distributed over the area thereof, wherein the injectors have an injection depth at a maximum as far as the subcutis of the animal or human to be treated.

Injection devices of the general kind set forth are known from EP 1 086 718 A1. Those injection devices serve for the injection of active substances or medicaments directly into the skin (intracutaneous injection) or they can be used for injection under the skin (subcutaneous injection). Intracutaneous injection obviates the metering problems known in relation to medical ointments and creams. In contrast to the ointments and creams in respect of which there is always the problem that it can be very difficult to control how much active substance and how quickly the active substance penetrates into the skin, the injection device of the general kind set forth provides that the active substance is introduced directly into the skin or into individual layers of the skin by means of a plurality of very short injectors. The active substances injected in that way are absorbed by the skin substantially better than ointments and creams which are applied to the skin. It is possible to forego the complicated gallenetics for preparation of the active substances, which is necessary for administration of active substances in the form of ointments and creams. It is also known from EP 1 086 718 A1 to apply anaesthetics by means of the injection device of the general kind set forth. The disadvantage of the variants of the injection device shown in EP 1 086 718 A1 however is that injection devices can still only be used by trained personnel safely and in particular under adequate conditions in terms of hygiene, which are to be created beforehand.

Therefore the object of the present invention is to provide an injection device for intracutaneous administration of at least an anaesthetic, in which application is further simplified along the lines such that it can also be implemented by non-specialist and untrained personnel.

In accordance with the invention that is achieved in that the medical injection device has a disinfecting agent on the side of the base surface on which the injectors are arranged.

The injection device according to the invention can thus be used to provide for local numbing of the human or animal to be treated, by means of the anaesthetic present in the injection device, in which respect in accordance with the invention disinfecting of the region of the skin which is to be treated is automatically effected at the same time. As a result of that the injection device according to the invention can also be used by untrained personnel and also outside hygienic treatment rooms, quickly and with an adequate level of hygiene. For that purpose, in the simplest alternative configurations, the injection device only has to be pressed against the skin of the animal or human to be treated. As the anaesthetic, it is possible to administer, in the widest sense, all medicaments and/or opioids acting on the nervous system, and other types of anaesthetics. In that respect, the injection can be effected specifically and targetedly into various layers of the skin, controlled by the length of the injectors. It is to be noted in that respect that an injection device according to the invention can be used to inject anaesthetics and preferably local anaesthetics into the epidermis in a pain-free manner as generally there are no pain-sensitive nerves present in the epidermis. That is particularly desirable as preparatory local numbing for small interventions, such as for example a more deeply extending injection with a syringe and injection needle. Thus a process for the injection of at least one active substance provides that firstly a given region of the animal or human to be treated is numbed with the medical injection device and then an injection is administered with a syringe, preferably in the numbed region.

In a particularly advantageous development of the invention, provided for that use is a medical set which comprises at least one sterile-packaged, already filled or empty injection syringe having an injection needle or only a sterile-packaged injection needle packaged in a sterile condition together with at least one medical injection device.

A desirable variant provides that the injectors and/or the base surface are wetted with disinfecting agent and/or anaesthetic. This embodiment has advantages in particular when only small amounts of anaesthetic are to be injected into the skin or individual layers of the skin (intracutaneously) and/or just under the skin (subcutaneously). In that case, when the injectors penetrate into the skin the anaesthetic is delivered thereto in a simple manner while the disinfecting agent is equally automatically applied to or introduced into the skin and provides for adequate disinfecting of the region of the skin being treated.

A point of particular advantage in the medical injection device is that the injectors are covered prior to use by a compressible guard or protective layer which can be penetrated by the injectors. In that respect favourable variants provide that the protective layer has a sponge and/or cellular material and/or fleece. In that respect the penetrable protective layer has essentially two functions. On the one hand the injectors are to be concealed in the protective layer prior to use in order to prevent unwanted injections or to prevent them from being prematurely destroyed. On the other hand the protective layer can be saturated with anaesthetic or disinfecting agent. In that case, the injectors remain until just before the injection is administered in the protective layer which is saturated with anaesthetic and/or disinfecting agent, and are accordingly constantly wetted thereby. That prevents the injectors from undesirably drying out. In that situation however the protective layer can be well compressed in such a way that the injectors reach their intended injection depth when the injection device is applied.

Another advantageous configuration provides that the injectors are covered by a cover means for storage and/or for transportation of the medical injection device as the cover means initially also affords protection for the injectors from unwanted injections or from damage. In a desirable development of this configuration it is provided that the cover means is at least partially filled with a disinfecting agent and/or an anaesthetic and the injectors in the covered condition dip into the disinfecting agent and/or the anaesthetic. That also ensures that the disinfecting agent and/or anaesthetic which adheres in the injectors and the base surface does not dry out, but the injectors have disinfecting agent and/or anaesthetic flowing therearound, until just before the injection.

An advantageous variant provides that the anaesthetic is a local anaesthetic. In that respect advantageous variants in turn provide that the local anaesthetic contains at least one of the substances cocaine, procaine, licoaine, prilocaine, benzocaine, tetracaine, cinchocaine, dperodon, dyclanine, mepivacaine-1, mepivacaine-2, prilocaine or pramocaine.

In regard to the disinfecting agent used in the medical injection device alcohol and/or hydrogen peroxide represent desirable options.

In regard to the injectors an advantageous embodiment provides that at least one injector is in the form of a solid needle or a partially flexible solid bristle. In that alternative configuration the solid needles or bristles are externally wetted with disinfecting agent and anaesthetic. This variant is particularly desirable when only small amounts of anaesthetic are to be administered. In addition, in this embodiment, continuous wetting of the injectors prior to the injection can be ensured either by a liquid supply of disinfecting agent in a cover means, as already mentioned above, or by means of a suitable protective layer.

For the purposes of administering larger amounts of anaesthetics a desirable embodiment provides that at least one injector is in the form of a hollow needle or a partially flexible hollow bristle. In this case in an advantageous development it can be provided that the medical injection device has at least one reservoir for the anaesthetic, wherein the reservoir is in hydraulic communication with at least one hollow needle or at least one partially flexible hollow bristle, wherein the anaesthetic can be injected into the skin through the hollow needle or the partially flexible hollow bristle. In that way even relatively large amounts of anaesthetic, which can be controlled by way of the volume of the reservoir, can be introduced into the skin or defined layers of the skin. In that respect it is once again desirable for the injectors and/or groups of injectors to involve different injection depths if the anaesthetic or anaesthetics is or are to be introduced into different layers of the skin. A very specific and targeted application of anaesthetics into defined layers of the skin, in defined amounts, with at the same time simplicity of operation, can also be effected by way of the choice of suitable reservoir sizes and appropriate injection depths. The number of injectors per injection depth and the way in which they are distributed over the area of the base surface can be respectively adapted to the medical function to be performed in each case.

Particularly for using the medical injection device away from sterile rooms or for the transportation of the medical injection device, it is particularly desirable for it to be packed in a sterile condition in a preferably tear-open sealed enclosure.

Besides the administration of anaesthetics, it can also be provided in a desirable development of the medical injection device that it also has at least one further active substance in addition to the anaesthetic and disinfecting agent. Very many different active substances may be considered here, some examples in that respect being anti-inflammatories, non-stearoidal anti-rheumatics, hormones, hormone antagonists and hormone argonists, cardiovascular medicaments and/or medicaments which act on the blood, preferably anti-coagulants, or also diagnostic substances or substances which do not act directly such as for example photosensitisers.

Further features and details of the present invention will be apparent from the specific description set out hereinafter. In the drawings:

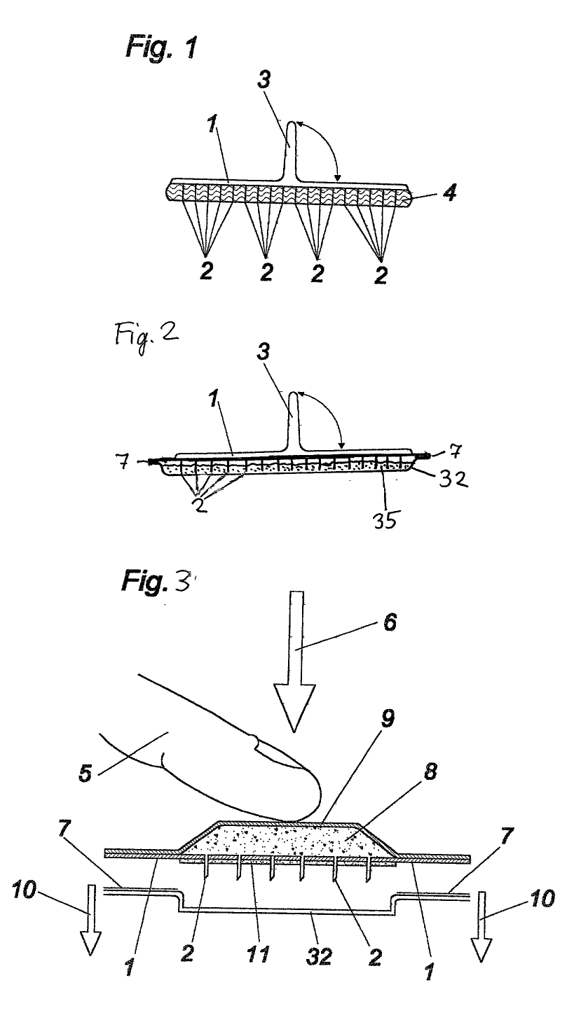

FIG. 1 shows an embodiment according to the invention with solid needles or solid bristles,

FIG. 2 shows an embodiment according to the invention with a disinfecting agent bath,

FIG. 3 shows an alternative configuration with a reservoir,

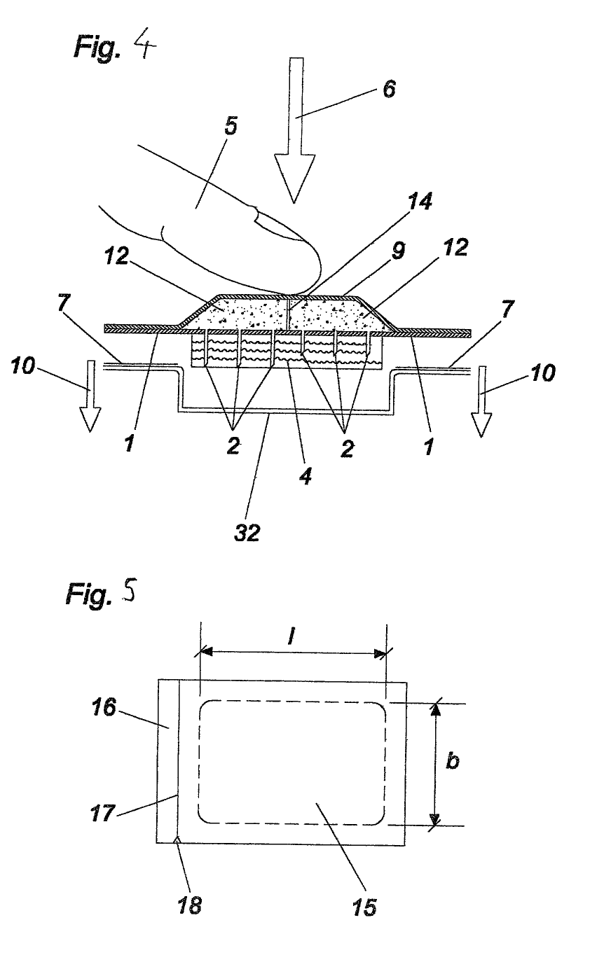

FIG. 4 shows a development of the configuration shown in FIG. 2,

FIG. 5 shows an example for sterile packaging,

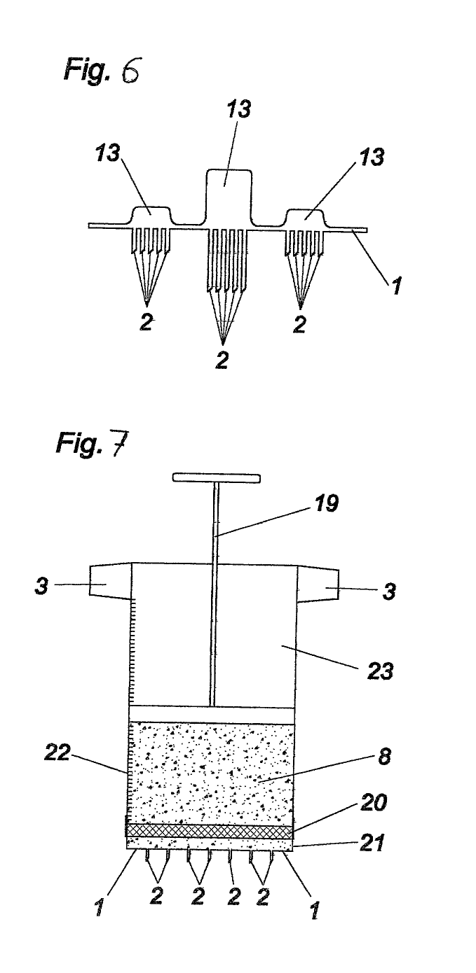

FIG. 6 shows an embodiment with three separate reservoirs and three groups of injectors of different lengths,

FIG. 7 shows a cylinder-plunger variant for multiple use,

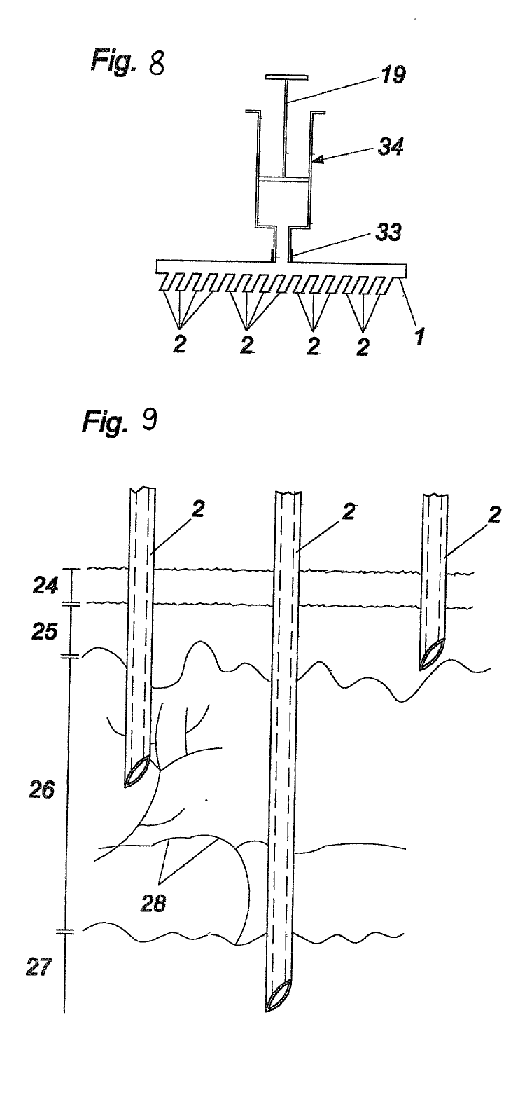

FIG. 8 shows an injection device as an attachment to a standard syringe,

FIG. 9 is a diagrammatic view for injection into different layers of the skin,

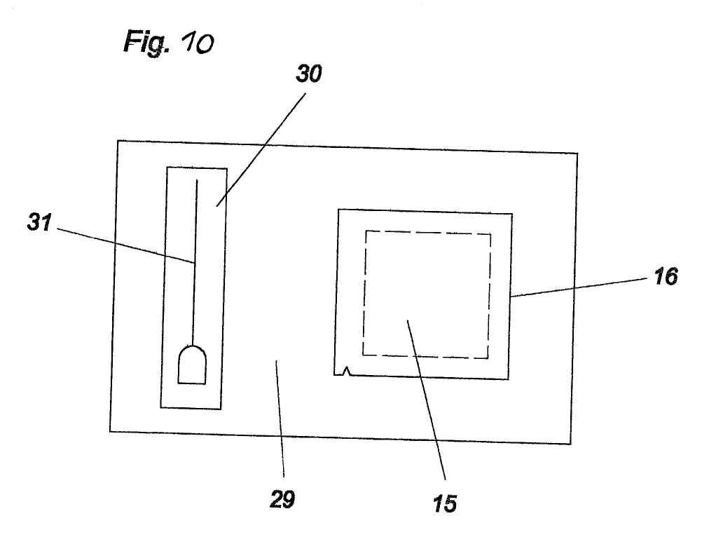

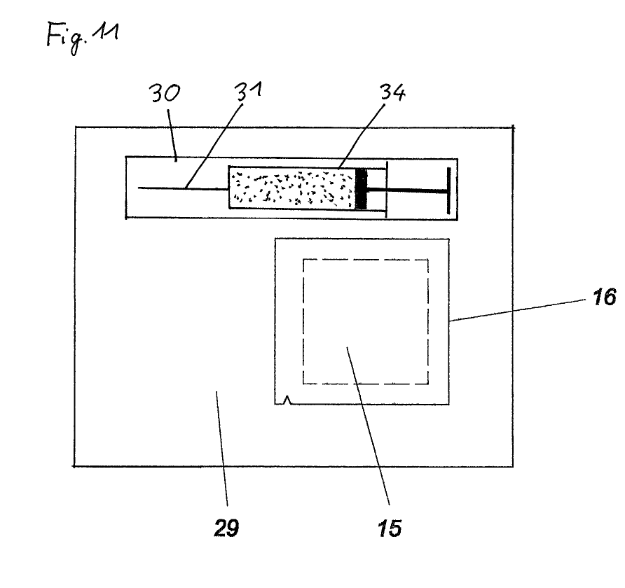

FIG. 10 shows a medical set with a sterile-packaged injection needle, and

FIG. 11 shows a medical set with a sterile-packaged, already filled injection syringe.

FIG. 1 shows a simple embodiment having a base surface 1, on which are arranged injectors 2 in the form of a solid needle or solid bristle. In accordance with the invention the injectors 2 are wetted with disinfecting agent and anaesthetic and before use, as illustrated, are concealed in a penetrable protective layer 4. In this arrangement the function of the protective layer 4 is to prevent unwanted injection prior to use of the injection device. On the other hand it can be made from absorbent material so that both disinfecting agent and also anaesthetic can be stored therein. That ensures optimum wetting of the injectors 2, even over relatively long periods of time. The penetrable protective layer 4 is desirably in the form of an absorbent layer, for example of cellulose, cotton, textile fabric or a combination thereof, or it is in the form of a sponge. The base surface 1 can preferably be made of plastic material or a metal-plastic laminate. The base surface is desirably of a fluid-impervious nature so that the anaesthetic is prevented from acting on the hand of the person administering the injection. Advantageous embodiments provide that the injection device has a handle 3 in the middle of the side of the base surface, which faces away from the injectors. That permits improved handling. The handle 3 is preferably flexible, that is to say it is designed to be folded over, so that the injection device can be packaged in a space-saving manner. The variant shown in FIG. 1 is suitable specifically for a use in which only little anaesthetic is to be administered as the needles or bristles are in the form of solid needles (this means needles which are not hollow) or solid bristles (this means bristles which are not hollow). Besides the variant shown in FIG. 1 the injectors 2 may also be of different lengths, individually or in group-wise manner, and thus may involve different injection depths. The protective layer 4 and the handle 3 are likewise to be viewed as being optional. The length of the injectors is matched to the layer of skin in which the injection is to be administered or whether the injection is to be made just under the skin.

FIG. 2 shows a variant in which injectors 2 are protected by a cover means 32 upon storage and transportation. The cover means 32 is fixed with the adhesive sealing material 7 to the base surface 1 of the medical injection device. Prior to the injection the cover means 32 is simply removed by being pulled off so that the injectors 2 then appear. During storage and transportation the injectors 2 are immersed in the disinfecting agent bath 35 present in the cover means 32. The disinfecting agent bath 35 may also contain anaesthetics. This configuration ensures that the injectors on the one hand are protected from physical damage and on the other hand are permanently uniformly wetted with disinfecting agent and/or anaesthetic. That ensures uniform quality of the injection even over relatively long periods of time. In addition the cover means 32 also affords protection from unwanted injections during handling and transportation.

FIG. 3 shows a variant according to the invention with which relatively large amounts of anaesthetic can be introduced into defined layers of the skin. In this case the injectors 2 are in the form of hollow needles which are in hydraulic communication with the reservoir 8. The reservoir 8 is sealed off by a flexible top side 9 which can be pressed in by the finger 5 in the direction indicated by the arrow 6. The sealing means 9 is fixed laterally from above on to the base surface 1. In accordance with the invention, arranged on the side of the injectors is a layer 11 with a disinfecting agent. For transportation purposes, it is particularly desirable for the injectors to be concealed by a protective covering (cover means) 32. This can be fitted removably to the base surface by an adhesive or an adhesive sealing material 7, and can cover over the injectors. The protective cover 32 thus prevents unwanted injection and prevents the injectors 2 and the reservoir 8 from drying out. The reservoir 8 can be in the form of a hollow chamber but it can also be in the form of a sponge having absorbent material. An amount of anaesthetic which is precisely suited to the respective use involved can be stored in the reservoir 8. Prior to use the protective cover 32 is removed. The injection device is then applied to the skin of the patient to be treated, in such a way that the injectors point towards the skin. The injectors are introduced into the skin of the patient by applying a slight pressure in the direction 10. The reservoir 8 is then emptied by pressing the finger 5 in the direction 6 on to the sealing enclosure 9. The anaesthetic stored in the reservoir 8 is injected through the hollow needles 12 into a defined layer of the skin of the patient. The layer of skin into which the injection is made can be very accurately controlled in that situation by the length of the injectors 2. The amount of medicament or active substance to be injected is determined by the size of the reservoir 8.

FIG. 4 shows a development of the structure shown in FIG. 3. In this case the reservoir 8 is subdivided into two reservoir portions 12. In addition, by way of example, the injectors 2 are subdivided into two groups involving different injection depths, which are in hydraulic communication with the various reservoirs 12. In addition, as shown in FIG. 1, the injectors can optionally again be concealed in a protective layer 4. This variant now affords the option of injecting different anaesthetics or additional active substances separately from each other into different layers of the skin. On the other hand it is now also possible to inject different amounts of anaesthetics or additional active substance into different layers of the skin, by virtue of the reservoir portions 12 being of different sizes. In that respect the example shown in FIG. 3 only represents an individual variant from a large number of different alternative configurations (not shown here) of the combination of various injectors, reservoirs and subdivisions thereof. In this respect there are virtually unlimited options in terms of combining those aspects in such a way as to be adapted to the respective use involved. Respective adaptation of the reservoir is possible by a suitable configuration of the flexible separating walls 14. The reservoir 8 or the reservoir portions 12 can in that case be in the form of sponges or in the form of hollow chambers.

FIG. 5 shows a sealing enclosure 16 of plastic or a plastic-metal laminate, which has a sterile internal volume 15 for receiving an injection device according to the invention, wherein the injection device according to the invention is already provided or impregnated in the ready condition with anaesthetic and possibly with additional active substances. The enclosure 16 has an incision 18 to make it easier to tear it open along the line 17. The enclosure 16 may also have other opening mechanisms such as for example incorporated threads or the like in order to make it easier to open the pack and to permit the injection device which is already ready for use to be taken out without having to touch the side which is later towards the skin. In this arrangement the injection device is of a length L and a width B, which are each preferably between 1 and 10 cm. Advantageous configurations of the injection device have the greatest extents of between 2 cm and 5 cm. It is to be emphasised in this respect that the sterile enclosure 16 shown in FIG. 4 is adapted to the respective size of the injection device to be accommodated therein.

FIG. 6 shows a variant of an injection device according to the invention in which three groups of injectors involve three different injection depths. Thus for example the group of injectors with the smallest depth of penetration can be provided for injection into the epidermis, the group with the injectors of medium length can be provided for injection into the dermis, and the group of long injectors can be provided for subcutaneous injection. In the illustrated example the individual groups of injectors 2 are connected to individual reservoir portions 13. In the illustrated example the reservoir portions 13 are of different volumes and are shown in the form of bags which are individually pressed in.

FIG. 7 shows a variant according to the invention for multiple use. In this arrangement the injection device is in the form of a syringe-like cylinder 21 with a plunger 19. The reservoir 8 is in communication with the injectors 2 by way of a porous separating layer 20. In the illustrated example the injectors are arranged on a replaceable lower portion 21. In that way the injection device can be equipped for example for adaptation of the lower portions 21 to different injector depths or for multiple use. In an advantageous development either a calibration bar or strip 22 or a latching means (not shown here) is provided for quantitative metering purposes. In the case of a latching means, when the plunger 19 is pressed in from one latch to the next, a defined amount of anaesthetic is discharged from the reservoir 8. In that case, for handling purposes, it is advantageous for the cylinder to be laterally provided with handles 3. In that arrangement the lower portion 21 can again have all the features shown in FIGS. 1, 2, 3, 4 and 6, for example different injector lengths or different distribution of the injectors over the surface area of the base surface. Furthermore all features shown in FIGS. 5 and 6 can be combined to adapt the injection devices to given areas of use.

FIG. 8 shows an alternative embodiment in which the injection device is in the form of an attachment for a standard syringe 34. In this case the standard syringe 34 is connected to the injection device by way of the flange 33. It is desirable in that respect for the insertion flange 33 to be designed in a standardised form so that any syringes which are known in the state of the art, for example one-trip or disposable syringes 34, can be used. In this embodiment the syringe 34 serves as a reservoir with the plunger 19 as the ejection device. In the embodiment shown in FIG. 7 numerous injectors 2 in the form of inclined hollow needles are arranged on the base surface 1. With those inclinedly arranged injectors 3, it is provided that all injectors are inclined in the same direction at the same angle and the injection device which is of that configuration is pushed laterally on to the skin until the base surface 1 bears against the skin. That once again ensures that the envisaged injection depth is precisely attained by the injectors 2. The injection device shown in FIG. 8 can be used in a particularly flexible manner by virtue of employing a standard syringe 34.

FIG. 9 shows by way of example a diagrammatic view illustrating the structure of the human skin with three injectors 2 which are diagrammatically illustrated in the form of hollow needles, which extend into different layers of the skin. The skin diagrammatically shown in FIG. 9 illustrates the stratum corneum 24, the epidermis 25, the dermis 26 and the subcutis 27. Also diagrammatically shown is a nerve strand 28 in the dermis. The epidermis has essentially no nerve strands running therethrough. Pain-free injection into the epidermis is therefore possible. Upon performing an injection into the epidermis of a human being an injection depth of between 0.1 mm and 0.2 mm is to be achieved by virtue of a suitable length for the injectors 2. For making an injection into the dermis 26 the length of the injectors should be between approximately 0.6 and 0.8 mm in the case of a human being. For injection into the subcutis (just under the skin) of a human being the injection depth of the injector or injectors 2 should be between about 1 and 1.5 mm. The example shown in FIG. 9 illustrates how the injector length can be adapted to the respective function to be performed and thus specific and targeted injection is achieved into defined layers of the skin.

FIG. 10 shows a medical set which comprises an injection needle 31 concealed in a sterile condition in a case or enclosure 30 and an injection device according to the invention which is packaged in a sterile pack 16 and the sterile internal volume 15 thereof. The medical set is disposed on a carrier 29 and closed in a sterile condition by a for example transparent cover means which is not shown here. This medical set is particularly advantageous as firstly the injection device according to the invention can be used for local numbing by means of an anaesthetic or local anaesthetic in order then to implement a deep injection with the needle 31 in the numbed region. In that way the wide-spread anxiety about injections can be taken away from the patient as firstly local numbing is effected with the medical injection device which can be applied in a pain-free manner and then there is no longer any pain involved when administering the deep injection. For the purposes of applying the injection device it only has to be pressed flat against the skin or pushed laterally against the skin (in the case of inclined injectors) and then possibly also the reservoir is expelled. The correct injection depth is achieved automatically in that case.

FIG. 11 shows a variant of FIG. 10 in which a ready-filled standard syringe 34 with injection needle 31 is already included in the medical set.

Finally it is to be pointed out that the injectors in FIGS. 1 through 4 and FIGS. 6 through 8 are shown on an exaggerated scale and too wide, for the sake of enhanced clarity of the drawing. In addition, all features contained in the illustrated embodiments can be combined in any form. The base surfaces 1 of the various alternative configurations of the injection devices can be rigid or partially flexible, depending on the respective requirement involved. The injectors are generally to be as thin as possible. With presentday manufacturing technology, in that respect maximum thicknesses of between 0.3 mm and 0.4 mm, preferably between 0.2 mm and 0.1, are possible. In addition the medical injection device can be used to administer the most widely varying additional active substances, medicaments or substances which have a diagnostic action or which do not act directly, such as for example photosensitisers.

1. A medical injection device for intracutaneous administration of at least one anaesthetic, comprising a base surface, on one side of which a plurality of injectors are arranged distributed over the area thereof, wherein the injectors have an injection depth at a maximum as far as the subcutis of the animal or human to be treated, characterized in that the medical injection device has a disinfecting agent on the side of the base surface on which the injectors are arranged.

2. The medical injection device as set forth in claim 1 characterized in that the injectors are injections needles.

3. The medical injection device as set forth in claim 1 characterized in that the injectors and/or the base surface are wetted with disinfecting agent and/or anaesthetic.

4. The medical injection device as set forth in claim 1 characterized in that prior to use the injectors are covered by a compressible protective layer which the injectors can penetrate.

5. The medical injection device as set forth in claim 4 characterized in that the protective layer is made of one or materials of the group consisting of a sponge, cellulose and fleece.

6. The medical injection device as set forth in claim 4 characterized in that the compressible protective layer contains a disinfecting agent and/or an anaesthetic.

7. The medical injection device as set forth in claim 6 characterized in that the compressible protective layer is preferably impregnated with a liquid disinfecting agent and/or anaesthetic.

8. The medical injection device as set forth in claim 1 characterized in that the injectors are covered by a cover means for storage and/or for transportation of the medical injection device.

9. The medical injection device as set forth in claim 8 characterized in that the cover means is at least partially filled with a disinfecting agent and/or an anaesthetic and the injectors dip in the covered condition into the disinfecting agent and/or the anaesthetic.

10. The medical injection device as set forth in claim 1 characterized in that the anaesthetic is a local anaesthetic.

11. The medical injection device as set forth in claim 1 characterized in that the local anaesthetic contains at least one of the substances cocaine, procaine, licoaine, prilocaine, benzocaine, tetracaine, cinchocaine, dperodon, dyclanine, mepivacaine-1, mepivacaine-2, prilocaine or pramocaine.

12. The medical injection device as set forth in claim 1 characterized in that the disinfecting agent contains alcohols and/or hydrogen peroxide.

13. The medical injection device as set forth in claim 1 characterized in that at least one injector is in the form of a solid needle or an at least partially flexible solid bristle.

14. The medical injection device as set forth in claim 1 characterized in that at least one injector is in the form of a hollow needle or an at least partially flexible hollow bristle.

13. The medical injection device as set forth in claim 14 characterized by at least one reservoir for the anaesthetic, the reservoir being in hydraulic communication with at least one hollow needle or at least one partially flexible hollow bristle, wherein the anaesthetic can be injected into the skin through the hollow needle or through the partially flexible hollow bristle.

16. The medical injection device as set forth in claim 1 characterized in that the injectors or groups of injectors involve different injection depths.

17. The medical injection device as set forth in claim 1 characterized in that it is packaged in a sterile condition in a sealing enclosure.

18. The medical injection device as set forth in claim 1 characterized in that it also has at least one further active substance in addition to the anaesthetic and disinfecting agent.

19. A medical set including
at least one sterile-packaged injection needle, and
at least one medical injection device as set forth in claim 1.

20. A medical set including
at least one sterile-packaged, already filled or empty injection syringe with or without an injection needle, and
at least one medical injection device as set forth in claim 1.

21. A method of injecting at least one active substance into the human or animal body characterized in that firstly a given region of the skin of the animal or human to be treated is numbed with a medical injection device having a plurality of injectors for intracutaneously administering at least one anaesthetic, and then an injection is applied with the syringe in the numbed region.

22. The method of claim 21 being performed by using the medical set as set forth in one of claims 17 and 18.

23. The method of claim 21 being performed by using the medical injection device of claim 1.
